(19) United States
(12) Patent Application Publication
(10) Pub. No.: US 2015/0306258 A1
(43) Pub. Date: Oct. 29, 2015

(54) PLASMA STERILIZATION OF CORNEAL TISSUE

(71) Applicants: James Ferrell, Stow, OH (US); Sameer Kalghatgi, Copley, OH (US); Tsung-Chan Tsai, Cuyahoga Falls, OH (US); Daphne Pappas Antonakas, Hudson, OH (US); Robert L. Gray, Hudson, OH (US)

(72) Inventors: James Ferrell, Stow, OH (US); Sameer Kalghatgi, Copley, OH (US); Tsung-Chan Tsai, Cuyahoga Falls, OH (US); Daphne Pappas Antonakas, Hudson, OH (US); Robert L. Gray, Hudson, OH (US)

(73) Assignee: EP TECHNOLOGIES LLC, Akron, OH (US)

(21) Appl. No.: 14/696,897

(22) Filed: Apr. 27, 2015

Related U.S. Application Data
(60) Provisional application No. 61/985,839, filed on Apr. 29, 2014.

(51) Int. Cl. A61L 2/00

(52) U.S. Cl. CPC A61L 2/0082

(57) ABSTRACT

Exemplary methods of treating tissue that has been harvested for transplant are disclosed herein. An exemplary method of preparing corneal tissue for transplant includes obtaining corneal tissue that has been harvested for transplanting and exposing the corneal tissue to a plasma-activated fluid for a period of time.

### RELATED APPLICATIONS

This non-provisional utility patent application claims priority to and the benefits of U.S. Provisional Patent Application Ser. No. 61/985,839 filed on Apr. 29, 2014 and entitled PLASMA STERILIZATION OF TISSUE. This application is incorporated herein by reference in its entirety.

### TECHNICAL FIELD

The present invention relates generally to methods and apparatuses for sterilizing transplant tissue, such as corneal tissue and more particularly to killing or deactivating bacteria or fungi on transplant tissue.

### BACKGROUND OF THE INVENTION

Bacterial and fungal infections associated with tissue transplantation create serious medical issues that may result in complications and even death or rejection of the tissue transplant. Contamination of the tissue may arise from an infected donor, during tissue removal, from cadaveric donors, from the processing environment, and from contaminated supplies and reagents used during processing. Some tissues are shipped in a storage media designed to preserve and protect tissue. For example, corneal tissue is shipped in Optisol®. A problem with this type of storage media is that Optisol® has no long term antimicrobial efficacy against any microorganism including resistant bacterial strains. In addition, fungi or viruses are not killed or deactivated by Optisol®.

### SUMMARY

Exemplary methods of sterilizing cornel tissue that has been harvested for transplant are disclosed herein. An exemplary method of preparing corneal tissue for transplant includes obtaining corneal tissue that has been harvested for transplanting and exposing the tissue to a gas plasma-activated fluid for a period of time.

Another exemplary method includes obtaining corneal tissue harvested for transplanting and applying a gas plasma-activated liquid or plasma jet to one or more sides of the corneal tissue for a predetermined period of time.

Another exemplary method of decontaminating corneal tissue includes obtaining corneal tissue harvested for transplanting and applying a gas plasma-activated liquid or plasma jet to one or more sides of the corneal tissue for a predetermined period of time sufficient to sterilize the corneal issue.

Another exemplary method of decontaminating corneal tissue includes obtaining corneal tissue harvested for transplanting and applying a gas plasma-activated liquid or plasma jet to one or more sides of the corneal tissue for a period of time sufficient to reduce the bacterial content by more than 99.999%.

Another exemplary method of decontaminating tissue includes obtaining corneal tissue harvested for transplanting and applying a plasma-activated liquid or plasma jet to one or more sides of the corneal tissue for a period of time sufficient to reduce the fungal content by more than 99.999%.

### BRIEF DESCRIPTION OF THE DRAWINGS

These and other features and advantages of the present invention will become better understood with regard to the following description and accompanying drawings in which:

FIG. 1 is an exemplary embodiment of tissue harvested for transplanting;

FIGS. 2A and 2B are schematic views of an exemplary embodiment of a gas plasma based tissue sterilization system;

FIG. 3 is an schematic view of an exemplary embodiment of a plasma-activated liquid based tissue sterilization system; and

FIGS. 4A and 4B are schematic views of an exemplary embodiment of a plasma-activated mist or plasma-activated vapor based tissue sterilization system.

### DETAILED DESCRIPTION

Gas plasmas, or ionized gases, have one or more highly energetic free electrons that are not bound to an atom or molecule. Plasmas are composed of significant concentrations of highly energetic and reactive species. These species have sufficient energy to stimulate rapid chemical reactions in localized environments that can trigger a chain reaction or “avalanche” of additional reactions.

Non-thermal plasmas, or cold plasmas, contain free electrons. Unlike thermal plasmas, the temperature of the free electrons in non-thermal plasmas is greater than the temperature of the ions and heavy neutral atoms within the plasma. The energy from the free electrons may be transferred to additional plasma components creating additional ionization, excitation and/or dissociation processes. Liquid that interacts with plasma becomes “activated” and is referred to herein as plasma-activated liquid, and in some embodiments is referred to as plasma-activated water.

In some embodiments, plasmas may contain superoxide anions [O2.-], which react with H+ in acidic media to form hydroperoxy radicals, HOO., which is a powerful antimicrobial: [O2.-]+[H+]→[HOO.]. Other radical species may include ONOO⁻, OH. and NO., known for their antimicrobial properties. Plasma-activated water may contain concentrations of one or more of peroxynitrite, H₂O₂, nitrates, and nitrites.

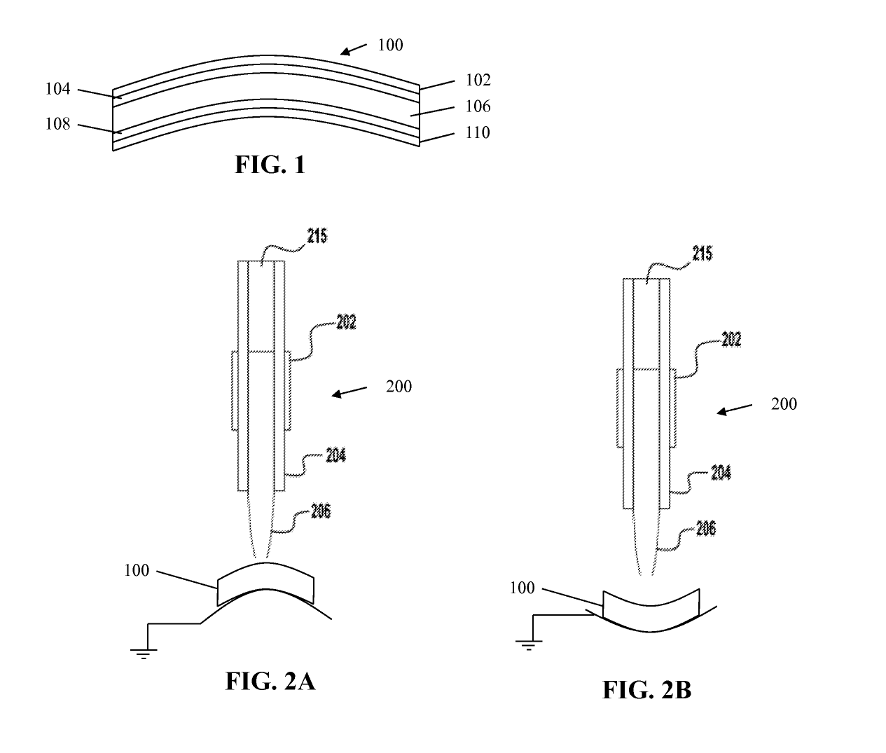

FIGS. 1 illustrates the layers of an exemplary tissue suitable for plasma sterilization. The exemplary tissue 100 is corneal tissue. Corneal tissue is made up of a number of layers, the epithelium layer 102, Bowman's layer 104, stroma 106, Descemet's membrane 108 and the endothelium layer 110. FIG. 2A illustrates an embodiment for treating the epithelial surface of a cornea and FIG. 2B illustrates an embodiment for treating the endothelial surface of a cornea 100.

The experiments detailed below utilized various fluids for sterilizing tissue. Some experiments utilized plasma jet system, some utilized plasma-activated liquid and some utilized plasma-activated mist or vapor. FIGS. 2A and 2B illustrate an exemplary embodiment of a plasma sterilization system 200 for sterilizing tissue 100 that utilized plasma gas. The exemplary plasma sterilization system 200 includes a non-thermal plasma generator 201 that includes a high voltage tubular electrode 202 and a fused quartz or borosilicate glass tube 204. Plasma generator 201 is a floating-electrode dielectric barrier discharge (DBD) plasma generator that generates a plasma “jet” 206.

Plasma generator 201 includes a gas feed 215. Exemplary gases that may be used to feed the plasma jet include He, He+O₂, N₂, He+N₂, air, He+air, Ar, Ar+O₂, Ar+N₂, and the like. In addition, combinations of two or more of the exemplary gasses may be used, such as, for example He, N₂ and O₂. Gases resulting from the evaporation of liquid solutions can also be used. Examples of vaporized liquids may include water, ethanol, organic solvents and the like. These vaporized liquids may be mixed with additive compounds. The evaporated liquids and additives may be used with the gases identified above in various concentrations or without the gases. The physical and chemical properties and concentration of the liquids, solvents and additives being utilized should be taken into account so as not to cause and damage to the applied tissue. Helium was used in plasma jet setups disclosed herein and room air was used for dielectric barrier discharge set ups and for plasma activated fluid set ups.

Plasma generator 201 includes a power supply, not shown. The power supply is a high voltage supply and may have a number of different wave forms, such as, for example, a constant, ramp-up, ramp-down, pulsed, picosecond pulsed, nanosecond pulsed, microsecond pulsed, square, sinusoidal, random, in-phase, out-of-phase, and the like. In the exemplary embodiments, the power supply was a sinusoidal power supply. The plasma 206 was generated by applying a sinusoidal voltage waveform. The particular settings for the power supply are discussed below with respect to certain experiments. During operation, the plasma jet 206 was in direct contact with the tissue.

For larger treatment areas, electrode configurations consisting of multiple plasma jets or larger area flat electrodes (not shown) may be used. In the case of more complex 3D surfaces, a controlled plasma module (not shown) may move around a stationary target or the surface to be exposed to the plasma may be placed on a movable stage. In some embodiments, one or more plasma jets can be attached to a robotic arm that is programmed to move in a manner that exposes one or more target areas to a plasma plume or jet. In some embodiments, shape conformable electrodes may be programmed to take the shape of the tissue to be treated followed by treatment with plasma-activated liquid, plasma-activated mist, plasma-activated vapor, plasma plumes, or plasma jets.

In some embodiments it is beneficial to generate non-thermal plasma using He, Ar, Ne, Xe and the like, air, or mixtures of inert gases with small percentage (0.5%-20%) of other gases such as O₂ and N₂ and mixtures of inert gases with vaporized liquids including water, ethanol, isopropyl alcohol, n-butanol, with or without additives and the like.

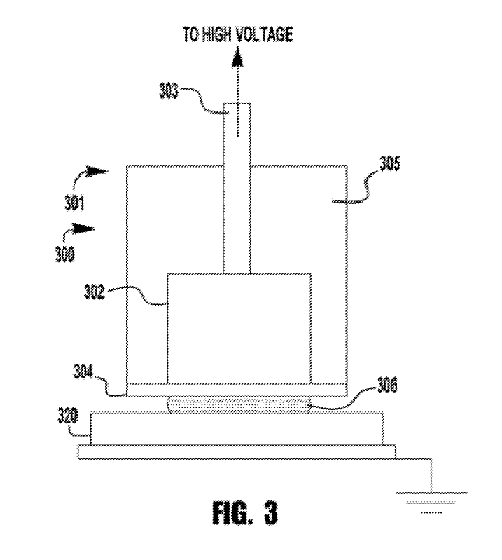

FIG. 3 illustrates another exemplary plasma treatment system 300 that utilized a plasma-activated liquid. Plasma treatment system 300 includes a plasma generator 301. Plasma generator 301 includes a high voltage wire 303 connected to an electrode 302 on a first end and a high voltage power supply (not shown) on the second end. Suitable high voltage supplies are described above. A dielectric barrier 304 is located below the high voltage electrode 302. In addition, the high voltage electrode 302 is located within a housing 305. Plasma generator 301 is a non-thermal direct barrier discharge (DBD) generator. Plasma 306 is generated by the plasma generator 301. FIG. 3 also includes container 320 for holding a fluid, such as, for example, water, to be activated.

Direct plasma 306 was generated by applying a voltage having a sinusoidal wave form to the electrode 302. The settings for the power supply and the time of exposure of the fluid to the plasma to activate the fluid is described below with respect to certain experiments.

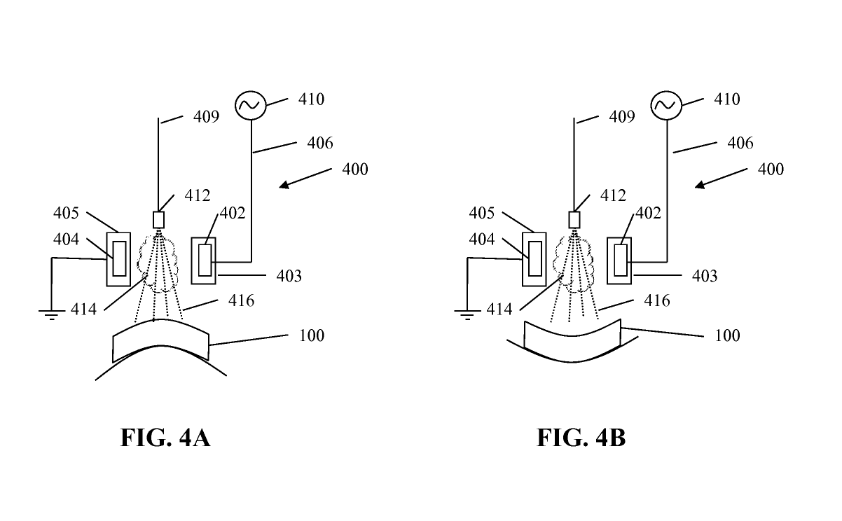

FIGS. 4A and 4B illustrate an exemplary embodiment of a plasma sterilization system 400 that utilized a plasma-activated mist or vapor to sterilized tissue. In the exemplary embodiment, the tissue is corneal tissue. FIG. 4A is an exemplary embodiment for treating the epithelial surface of the corneal tissue and FIG. 4B is an exemplary embodiment for treating the endothelial surface. Plasma sterilization system 400 activates and applies the mist or vapor to tissue 100. Plasma sterilization system 400 includes a passage 409 for delivering an atomized fluid 412. In one embodiment, the fluid is distilled water. An atomizer 412 creates a vapor or mist. Atomizer 412 creates a large surface area around the mist or vapor droplets. The large surface area allows for a quicker activation of the fluid. The mist or vapor is directed between electrodes 402 and 404.

Electrode 402 is connected to a high voltage source 410 and is surrounded by a dielectric barrier 403. Electrode 404 may also be at least partially surrounded by a dielectric barrier 405. Dielectric barriers 403, 405 prevent arcing between electrode 402 and electrode 404, which is connected to a ground. Dielectric barriers 403, 405 may include, for example, polymers, plastic, glass, ceramics or other known dielectric barrier materials. High voltage source 410 is connected to electrode 402 by cable 406. High voltage source 410 may have an output of, for example, between about 1 kV to 30 kV at between about 0.05 kHz and 30 kHz. In one embodiment, the distance between electrodes 402 and 404 is between about 2 mm and several centimeters.

When electrode 402 is energized, non-thermal plasma 414 is generated between the electrodes 402, 404 by ionizing the gas located between the electrodes 402, 404. Fluid travels under pressure through conduit 409 and through atomizer 412. Atomizer 412 may be, for example, a piezoelectric element, an atomizing nozzle, an aerosol container containing the liquid with a pressurized gas or other mechanism that creates a mist or fine spray of fluid 416. The vapor, mist or fine spray of fluid 416 passes through the plasma 414 and becomes plasma-activated liquid, such as plasma-activated mist or plasma-activated vapor. The droplets in the plasma-activated mist or vapor 416 can be electrostatically charged so that they can become attracted to negatively charged, or grounded, or electrically floating, objects such as the tissue 100 (FIG. 1).

In addition, the properties of the activated fluid described above may be adjusted during the activation process itself by altering the gas that is ionized. For example, the gas that is ionized may be normal air, N₂, O₂, CO₂, He, Ar, Xe or combinations thereof

For the experiments detailed herein, the tissue sample number is located in column 1. Column 2 identifies the type of plasma sterilization system that was used. The power settings of the plasma power supply are identified in column 3. The power supply was a sinusoidal power supply. The power supply has a number of settings ranging from a scale 1 to a scale 60 that correspond to a voltage of 1 kV to 30 kV. The scale of the power setting is identified in column 3. In addition, column 3 includes the frequency settings and the duty cycle (“DC”). Also identified in column 3, for the mist or vapor plasma sanitization systems is the flow rate for the mist or vapor. The flow rate of the mist was equivalent to 2.33 ul per minute. In addition, column 3 includes a “gap” setting. For the mist plasma sanitization systems, the gap indicates the distance from the spout where plasma-activated mist left the electrode chamber to the surface of the tissue. For the plasma jet based sanitization systems, the gap indicates the distance from the end of the glass tube to the surface of the tissue. For the plasma-activated liquid sanitization systems, the gap indicates the distance from the end of the DBD electrode to the surface of the liquid being activated.

Column 4 indicates the exposure time, which is the amount of time the plasma sterilization system was used on the surface of the tissue. In the case of the liquid, the liquid, which in this case was water, was activated for 60 seconds.

Column 5 indicates the bacteria that survived the treatment and was able to be recovered. These data were then used to tabulate the overall log reduction seen in Column 7.

In the experiments illustrated in Table 1, human corneal tissue was gently rinsed with deionized water in order to remove any residual storage medium (Opti-Sol®) and was placed within humidified chambers to prevent tissue desiccation. Ten microliters of a bacterial culture (Escherichia coli ATCC 35150) with an initial concentration of ˜10⁹ colony formation units (CFU) per ml was pipetted onto the endothelial layer (face) of the corneal substrates. The bacteria remained on the corneal tissue for ˜30 minutes within humidified chambers. Each corneal tissue sample was exposed to non-thermal plasma in a regimen as described in Table 1.

The negative control can be described as a sample where the tissue was contaminated with bacteria, but was unexposed to any plasma sterilization system or any other antimicrobial intervention. All experimental samples were placed in buffered saline prior to bacterial suspension dilution. Diluted cultures were then plated on nutrient agar plates and incubated at 37° C. for ˜16 hours. Any surviving bacteria were determined by direct enumeration of bacterial colonies that grew on the agar plates. Raw numbers of viable colonies were then used to calculate (based on the dilution of the samples) the logarithmic viability and reduction values seen in Table 1.

TABLE 1

| | | | Exposure | Surviving | |
|---|---|---|---|---|---|
| | Plasma | Plasma Power Supply | Time | Bacteria Log₁₀ | |
| Sample | Fluid | Settings | (seconds) | CFU/ml | Log₁₀ Reduction |
| 1 | Mist | Scale 15; f: 21.5 KHz; | 15 | 7.982 | 0.444 |
| | | DC = 100%; 5 mm gap; | | | |
| | | Mist Flow: Scale 1.5 | | | |
| 2 | Mist | Scale 15; f: 21.5 KHz; | 30 | 8.477 | −0.051 |
| | | DC = 100%; 5 mm gap; | | | |
| | | Mist Flow: Scale 1.5 | | | |
| 3 | Mist | Scale 15; f: 21.5 KHz; | 45 | 0.00 | 5.000 |
| | | DC = 100%; 5 mm gap; | | | (Complete Kill) |
| | | Mist Flow: Scale 1.5 | | | |
| 4 | Mist | Scale 15; f: 21.5 KHz; | 60 | 0.00 | 5.000 |
| | | DC = 100%; 5 mm gap; | | | (Complete Kill) |
| | | Mist Flow: Scale 1.5 | | | |
| 5 | Gas | Scale 8; f: 25 KHz; DC = | 5 | 7.260 | 1.166 |
| | | 100%; 5 mm gap | | | |
| 6 | Gas | Scale 8; f: 25 KHz; DC = | 5 | 8.477 | 0.949 |
| | | 100%; 5 mm gap | | | |
| 7 | Gas | Scale 8; f: 25 KHz; DC = | 5 | 7.491 | 0.935 |
| | | 100%; 5 mm gap | | | |
| 8 | Gas | Scale 8; f: 25 KHz; DC = | 15 | 0.000 | 5.000 |
| | | 100%; 5 mm gap | | | (Complete Kill) |
| 9 | Liquid | Scale 15; f: 21.5 KHz; | 30 | 7.919 | 0.507 |
| | | DC = 100%; 1.5 mm | | | |
| | | gap | | | |
| 10 | Liquid | Scale 15; f: 21.5 KHz; | 60 | 0.000 | 5.000 |
| | | DC = 100%; 1.5 mm | | | (Complete Kill) |
| | | gap | | | |
| 11 | N/A | Negative Control | | 8.427 | N/A |

As can be seen from Table 1, the settings for samples 3, 4, 8 and 10 resulted in complete kill, represented by an approximate 5.0 log reduction of E. coli ATCC 35150 according to the plasma application regimens for each of those respective samples. The actual calculated log reduction is closer to 7.0 logs, but the experimental design was limited to a maximum 5.0 logarithmic limit of detection count. The settings used with samples 4, 8, and 10 were expanded upon with additional experimentation.

In the experiments illustrated in Table 2, a 10 μl of Staphylococcus. aureus ATCC 33591 with an initial concentration of ˜10⁹ CFU/ml was pipetted onto the endothelial layer (face) for the corneal substrate. Handling, plasma exposure, and enumeration were performed as described previously.

TABLE 2

| | | | Exposure | Surviving | |
|---|---|---|---|---|---|
| | Plasma | Plasma Power Supply | Time | Bacteria Log₁₀ | |
| Sample | Fluid | Settings | (seconds) | CFU/ml | Log₁₀ Reduction |
| 1 | Mist | Scale 15; f: 21.5 KHz; | 60 | 0 | 5.000 |
| | | DC = 100%; 5 mm gap; | | | (Complete Kill) |
| | | Mist Flow: Scale 1.5 | | | |
| 2 | Mist | Scale 15; f: 21.5 KHz; | 60 | 0 | 5.000 |
| | | DC = 100%; 5 mm gap; | | | (Complete Kill) |
| | | Mist Flow: Scale 1.5 | | | |
| 3 | Liquid | Scale 15; f: 21.5 KHz; | 60 | 0 | 5.000 |
| | | DC = 100%; 1.5 mm | | | (Complete Kill) |
| | | gap | | | |
| 4 | Liquid | Scale 15; f: 21.5 KHz; | 60 | 0 | 5.000 |
| | | DC = 100%; 1.5 mm | | | (Complete Kill) |
| | | gap | | | |
| 5 | Gas | Scale 15; f: 21.5 KHz; | 15 | 0 | 5.000 |
| | | DC = 100%; 5 mm gap | | | (Complete Kill) |
| 6 | Gas | Scale 15; f: 21.5 KHz; | 15 | 0 | 5.000 |
| | | DC = 100%; 5 mm gap | | | (Complete Kill) |
| 7 | N/A | Negative Control | | 6.988 | N/A |
| | | Average (n = 2) | | | |

As can be seen from Table 2, the settings for all of the samples that were treated with plasma sanitization systems resulted in complete kill, represented by an approximate 5.0 log reduction of S. aureus.

In the experiments illustrated in Table 2, a 10 ul aliquot of Staphylococcus aureus ATCC 33591 with an initial concentration of ˜10⁹ CFU/ml was pipetted onto the endothelial layer (face) for the corneal substrate. Handling, plasma exposure, and enumeration were performed as described previously.

TABLE 3

| | | | Exposure | Surviving | |
|---|---|---|---|---|---|
| | Plasma | Plasma Power Supply | Time | Bacteria Log₁₀ | |
| Sample | Fluid | Settings | (seconds) | CFU/ml | Log₁₀ Reduction |
| 1 | Mist | Scale 15; f: 21.5 KHz; | 60 | 0 | 8.074 |
| | | DC = 100%; 5 mm gap; | | | (Complete Kill) |
| | | Mist Flow: Scale 1.5 | | | |
| 2 | Mist | Scale 15; f: 21.5 KHz; | 60 | 0 | 8.074 |
| | | DC = 100%; 5 mm gap; | | | (Complete Kill) |
| | | Mist Flow: Scale 1.5 | | | |
| 3 | Liquid | Scale 15; f: 21.5 KHz; | 60 | 5.591 | 2.483 |
| | | DC = 100%; 1.5 mm | | | |
| | | gap | | | |
| 4 | Liquid | Scale 15; f: 21.5 KHz; | 60 | 5.322 | 2.751 |
| | | DC = 100%; 1.5 mm | | | |
| | | gap | | | |
| 5 | Gas | Scale 15; f: 21.5 KHz; | 15 | 3.279 | 4.795 |
| | | DC = 100%; 5 mm gap | | | |
| 6 | Gas | Scale 15; f: 21.5 KHz; | 15 | 5.279 | 2.795 |
| | | DC = 100%; 5 mm gap | | | |
| 7 | N/A | Negative Control | | 8.074 | N/A |
| | | Average (n = 2) | | | |

As can be seen from Table 3, the settings for all of the samples that were treated with plasma mist sanitization systems and one of the gas plasma sanitization systems resulted in complete kill, represented by an approximate 5.0 log reduction of S. aureus. In addition, although the fluid plasma sanitization systems and one of the gas plasma sterilization systems did not result in complete kill, in each case, there was a significant log reduction.

As can be seen from the above experiments, the plasma sanitization systems demonstrated efficacy in bacteria kill and deactivation.

In the experiments illustrated in Tables 3 & 4, 10 μl of the fungus Candida albicans (C. albicans) ATCC 20308 with an initial concentration of ˜10⁹ CFU/ml was pipetted onto the endothelial layer (face) of the corneal substrate. Handling, plasma exposure, and enumeration were performed as described previously.

TABLE 4

| | | | Exposure | Surviving | |
|---|---|---|---|---|---|
| | Plasma | Plasma Power Supply | Time | Bacteria Log₁₀ | |
| Sample | Fluid | Settings | (seconds) | CFU/ml | Log₁₀ Reduction |
| 1 | Mist | Scale 15; f: 21.5 KHz; | 60 | 0 | 5.000 |
| | | DC = 100%; 5 mm gap; | | | (Complete Kill) |
| | | Mist Flow: Scale 1.5 | | | |
| 2 | Mist | Scale 15; f: 21.5 KHz; | 60 | 0 | 5.000 |
| | | DC = 100%; 5 mm gap; | | | (Complete Kill) |
| | | Mist Flow: Scale 1.5 | | | |
| 3 | Liquid | Scale 15; f: 21.5 KHz; | 60 | 5.447 | 1.106 |
| | | DC = 100%; 1.5 mm | | | |
| | | gap | | | |
| 4 | Liquid | Scale 15; f: 21.5 KHz; | 60 | 0 | 1.001 |
| | | DC = 100%; 1.5 mm | | | |
| | | gap | | | |
| 5 | Gas | Scale 15; f: 21.5 KHz; | 15 | 5.996 | 1.655 |
| | | DC = 100%; 5 mm gap | | | |
| 6 | Gas | Scale 15; f: 21.5 KHz; | 15 | 6.101 | 5.000 |
| | | DC = 100%; 5 mm gap | | | (Complete Kill) |
| 7 | N/A | Negative Control | | 7.102 | N/A |
| | | Average (n = 2) | | | |

As can be seen from Table 4, the treatment conditions for all of the samples that were treated with plasma mist sanitization systems and one of the liquid plasma sanitization systems resulted in complete kill, represented by an approximate 7 log reduction of C. albicans. One of the liquid plasma sanitization systems did not result in complete kill, but did have a significant reduction in S. albicans. The gas-based plasma sanitization system reduced the C. albicans. As can be seen, the plasma sterilization systems demonstrated efficacy in killing and deactivating fungi. The plasma mist resulted in an average of a 5.000 log reduction The plasma jet resulted in an average 4.378 log reduction and plasma-activated liquid resulted in an average 1.054 log reduction in C. albicans.

An independent party, Lions Vision Gift eye bank, analyzed the corneal tissue after treatment with the plasma sanitization systems for damage. The analyzing included viability staining The results of the analysis demonstrated that the tissue was not damaged by the plasma sterilization processes.

While the present invention has been illustrated by the description of embodiments thereof and while the embodiments have been described in considerable detail, it is not the intention of the applicants to restrict or in any way limit the scope of the appended claims to such detail. Additional advantages and modifications will readily appear to those skilled in the art. For example, the sanitizing station 400 may be utilized in a shower, or portable shower system that may be set up to decontaminate persons or large objects on a site that has become contaminated by bacteria. Moreover, elements described with one embodiment may be readily adapted for use with other embodiments. Therefore, the invention, in its broader aspects, is not limited to the specific details, the representative apparatus and illustrative examples shown and described. Accordingly, departures may be made from such details without departing from the spirit or scope of the applicants' general inventive concept.

We claim:

1. A method of preparing corneal tissue for transplant comprising:
obtaining corneal tissue that has been harvested for transplanting;
exposing the harvested corneal tissue to a plasma-activated fluid for a period of time.

2. The method of claim 1 wherein the plasma-activated fluid is a gas.

3. The method of claim 2 wherein the gas is helium.

4. The method of claim 2 wherein the gas is air.

5. The method of claim 1 wherein the plasma-activated fluid includes water.

6. The method of claim 1 wherein the plasma-activated fluid is a mist.

7. The method of claim 1 wherein the plasma-activated fluid is a liquid.

8. The method of claim 1 wherein the exposure time is less than about 60 seconds.

9. The method of claim 1 wherein the exposure time is less than about 30 seconds.

10. The method of claim 1 wherein the exposure time is less than about 15 seconds.

11. The method of claim 1 wherein the exposure time is less than about 5 seconds.

12. A method of sterilizing corneal tissue comprising;
obtaining corneal tissue that has been removed from a body;
applying a plasma-activated fluid to one or more sides of the corneal tissue for a predetermined period of time.

13. The method of claim 12 wherein the predetermined time is greater than about 5 seconds

14. The method of claim 12 wherein the predetermined time is greater than about 10 seconds.

15. The method of claim 12 wherein the predetermined time is less than about 60 seconds.

16. The method of claim 12 wherein sterilizing the corneal tissue comprises killing or deactivating bacteria.

17. The method of claim 12 wherein sterilizing the corneal tissue comprises killing or deactivating fungus.

18. A method of treating harvested corneal tissue comprising;
obtaining corneal tissue harvested for transplanting;
applying a plasma-activated fluid to one or more sides of the harvested corneal tissue for a period of time sufficient to reduce the bacterial content by more than 99.999%.

19. A method of treating harvested corneal tissue comprising;
obtaining corneal tissue harvested for transplanting;
applying a plasma-activated fluid to one or more sides of the harvested corneal tissue for a period of time sufficient to reduce the fungal content by more than 99.999%.

20. The method of claim 19 wherein the period of time is less than about 60 seconds.
